(19) United States
(12) Patent Application Publication
(10) Pub. No.: US 2022/0265228 A1
(43) Pub. Date: Aug. 25, 2022

(54) RADIATION IMAGING SYSTEM, RADIATION IMAGING METHOD, IMAGE PROCESSING APPARATUS, AND STORAGE MEDIUM

(71) Applicant: CANON KABUSHIKI KAISHA, Tokyo (JP)

(72) Inventors: Yasutomo Shimizu, Tochigi (JP); Yuichi Ikeda, Shizuoka (JP)

(21) Appl. No.: 17/742,238

(22) Filed: May 11, 2022

Related U.S. Application Data
(63) Continuation of application No. PCT/JP2020/041695, filed on Nov. 9, 2020.

(30) Foreign Application Priority Data
Nov. 18, 2019 (JP) 2019-208330

(51) Int. Cl. A61B 6/04; G06T 19/00; G06T 7/70; A61B 6/00

(52) U.S. Cl. CPC A61B 6/04; G06T 19/00; G06T 7/70; A61B 6/463; A61B 6/54; G06T 2219/004; G06T 2207/20081; G06T 2207/30196; G06T 2210/41

(57) ABSTRACT

A radiation imaging system includes a first generation unit that generates, based on a first optical image of a subject acquired at a first time, a first processed image including at least one of information indicating a skeleton of the subject and information indicating a joint angle of the subject related to the first optical image, a second generation unit that generates, based on a second optical image of the subject acquired at a second time different from the first time, a second processed image including at least one of information indicating the skeleton of the subject and information indicating the joint angle of the subject related to the second optical image, and a display control unit that displays the first processed image and the second processed image in a superimposed manner on a display unit.

### CROSS-REFERENCE TO RELATED APPLICATIONS

This application is a Continuation of International Patent Application No. PCT/JP2020/041695, filed Nov. 9, 2020, which claims the benefit of Japanese Patent Application No. 2019-208330, filed Nov. 18, 2019, both of which are hereby incorporated by reference herein in their entirety.

### BACKGROUND OF THE INVENTION

### Field of the Invention

The present invention relates to a radiation imaging system, a radiation imaging method, an image processing apparatus, and a storage medium.

### Background Art

Conventionally, in imaging using a radiation imaging system in the medical field, there is a case where an imaging target part is captured in the same posture as in the past in a follow-up examination. In such a case, a user such as a doctor needs to determine the same posture based on an image captured in the past. Thus, it takes time to position the posture of a patient.

In recent years, in response to the above issue, the following configuration exists.

PTL 1 discusses a technique for attaching an optical camera to a radiation generating apparatus and storing an optical image captured by the optical camera when the radiation generating apparatus generates radiation, together with a radiation image and the imaging conditions of a patient. Then, the technique generates a guide image from the optical image and displays the guide image in a superimposed manner on an optical moving image displayed on a display device of an imaging system when the same patient is subsequently captured.

However, in a case where it is desirable to perform positioning while grasping a skeleton and joint angles of a patient, such as a case where a joint is captured, the mere superimposed display of an optical image of the patient inspected in the past as a guide may not necessarily be able to improve the reproducibility of the positioning.

### CITATION LIST

### Patent Literature

PTL 1: Japanese Patent Application Laid-Open No. 2014-117368

### SUMMARY OF THE INVENTION

The present invention is directed to improving the reproducibility of the positioning of the posture of a patient in imaging using a radiation imaging system.

A radiation imaging system for imaging a subject using radiation according to the present invention includes a first generation unit configured to generate, based on a first optical image of the subject acquired at a first time, a first processed image including at least one of information indicating a skeleton of the subject and information indicating a joint angle of the subject related to the first optical image, a second generation unit configured to generate, based on a second optical image of the subject acquired at a second time different from the first time, a second processed image including at least one of information indicating the skeleton of the subject and information indicating the joint angle of the subject related to the second optical image, and a display control unit configured to display the first and second processed images in a superimposed manner on a display unit.

Further features of the present invention will become apparent from the following description of exemplary embodiments with reference to the attached drawings.

### BRIEF DESCRIPTION OF THE DRAWINGS

FIG. 1 is a diagram illustrating an example of a system configuration of a medical image system according to a first exemplary embodiment.

FIG. 2 is a block diagram illustrating an example of a configuration of a radiation imaging control apparatus according to the first exemplary embodiment.

FIG. 3 is a block diagram illustrating an example of a configuration of a radiation imaging control apparatus according to the first exemplary embodiment.

FIG. 4 is a flowchart illustrating an example of processing steps of the radiation imaging control apparatus according to the first exemplary embodiment.

FIG. 5 is a diagram illustrating an example of a display configuration of a display unit when the radiation imaging control apparatus according to the first exemplary embodiment captures a patient.

FIG. 6 is a configuration diagram illustrating an example of patient information regarding the radiation imaging control apparatus according to the first exemplary embodiment.

FIG. 7 is a diagram illustrating an example of a display configuration of the display unit when the radiation imaging control apparatus according to the first exemplary embodiment captures the patient inspected in the same posture in the past.

FIG. 8 is a diagram illustrating an example of a configuration of a guide image setting screen of a radiation imaging control apparatus according to a second exemplary embodiment.

FIG. 9 is a diagram illustrating an example of a display configuration of the display unit when the radiation imaging control apparatus according to the second exemplary embodiment captures the patient inspected in the same posture in the past.

FIG. 10 is a block diagram illustrating an example of a configuration of a radiation imaging control apparatus according to a third exemplary embodiment.

FIG. 11 is a flowchart illustrating an example of processing steps of the radiation imaging control apparatus according to the third exemplary embodiment.

FIG. 12 is a diagram illustrating an example of a display configuration of a display unit when the radiation imaging control apparatus according to the third exemplary embodiment captures a patient.

### DESCRIPTION OF THE EMBODIMENTS

Desirable exemplary embodiments of a radiation imaging system according to the present invention will be described in detail below with reference to the attached drawings. The components described in the exemplary embodiments, however, are merely illustrative. The technical scope of the radiation imaging system according to the present invention is determined by the appended claims, and is not limited by the following individual exemplary embodiments. The present invention is not limited to the following exemplary embodiments. Various modifications (including the organic combinations of the exemplary embodiments) can be made based on the spirit of the present invention, and are not excluded from the scope of the present invention. That is, all the configurations obtained by combining the following exemplary embodiments and their variations are also included in the exemplary embodiments of the present invention.

### First Exemplary Embodiment

A radiation imaging system according to a first exemplary embodiment is a radiation imaging system that displays an optical image obtained by capturing a patient on a display device and enables positioning in radiation imaging while viewing the optical image, and is directed to improving positioning accuracy when an inspection is performed.

More specifically, while information indicating a skeleton of a patient as an inspection target and information indicating joint angles of the patient are displayed in a superimposed manner on an optical image (a current image) obtained by capturing the patient, information indicating the skeleton and information indicating the joint angles related to a past image obtained by capturing the patient in the same posture in a past are superimposed on the resulting image. An inspector can then perform positioning based on the information indicating the skeleton and the information indicating the joint angles related to the past image that are displayed in a superimposed manner and the information indicating the skeleton and the information indicating the joint angles displayed in a superimposed manner on the current image.

Although the present exemplary embodiment is described using as an example a form in which two pieces of information, namely information indicating the skeleton and information indicating the joint angles, are superimposed on an optical image, the two pieces of information do not necessarily need to be superimposed. Alternatively, at least one of the information indicating the skeleton and the information indicating the joint angles may be superimposed on the optical image. In a case where only either one of the two pieces of information is superimposed on the optical image, it is desirable that information to be superimposed on a current image and information to be superimposed on a past image should match each other. However, the information to be superimposed on the current image and the information to be superimposed on the past image do not need to completely match each other, and may partially match each other.

Although the present exemplary embodiment is described using a radiation imaging system as an example, a modality that performs imaging may be a magnetic resonance imaging (MRI) apparatus, a three-dimensional ultrasound imaging apparatus, or a photoacoustic tomography apparatus. That is, the present invention is applicable to any apparatus required to capture images under the same condition in the past and the present. Further, optical images obtained by capturing the patient as the inspection target do not necessarily need to be images acquired by a single image acquisition apparatus, and may be images obtained by using different image acquisition apparatuses.

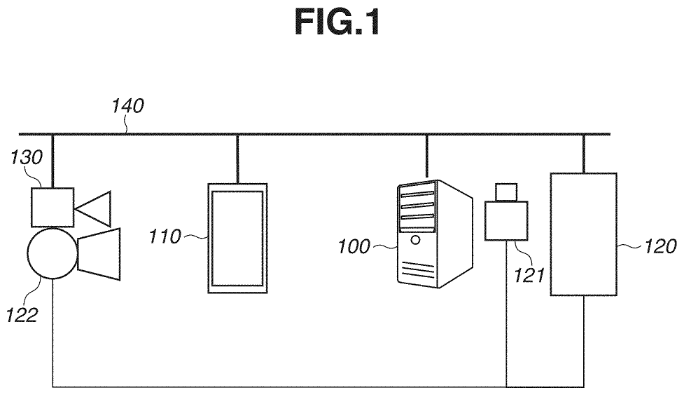

The configuration of the system according to the present exemplary embodiment is described below with reference to FIGS. 1 to 3.

FIG. 1 is an example of the configuration of the entirety of the radiation imaging system according to the present exemplary embodiment. This system includes, via a network 140, a radiation imaging control apparatus 100, a radiation imaging apparatus 110, a radiation generating apparatus 120, and an image acquisition apparatus 130. The network 140 may be a wired network or a wireless network.

The radiation imaging control apparatus 100 is composed of an information processing apparatus, such as a computer that communicates with the radiation imaging apparatus 110 and controls radiation imaging. The radiation imaging control apparatus 100 also communicates with the radiation generating apparatus 120 and acquires information when the radiation generating apparatus 120 emits radiation. Further, the radiation imaging control apparatus 100 communicates with the image acquisition apparatus 130, controls the image acquisition apparatus 130, and acquires an image captured by the image acquisition apparatus 130.

The radiation imaging apparatus 110 includes a flat-panel detector (FPD) and generates a radiation image based on incident radiation. The radiation imaging apparatus 110 transitions to a state where the radiation imaging apparatus 110 can perform imaging based on an instruction from the radiation imaging control apparatus 100. The radiation imaging apparatus 110 then performs radiation imaging in synchronization with the radiation generating apparatus 120 under a predetermined imaging condition set by a user, thereby generating an image based on radiation emitted from the radiation generating apparatus 120. That is, the radiation imaging apparatus 110 corresponds to an example of an acquisition unit configured to acquire a radiation image based on radiation emitted to a subject.

The number of radiation imaging apparatuses 110 is not limited to one, and a configuration may be employed in which a plurality of radiation imaging apparatuses is used.

The radiation generating apparatus 120 detects a radiation emission instruction from an exposure switch 121, and causes a tubular lamp 122 to generate radiation based on emission information set using a user input device (not illustrated) such as an operation panel for receiving a user operation.

The image acquisition apparatus 130 performs imaging based on an instruction from the radiation imaging control apparatus 100, thereby acquiring an image. In the present exemplary embodiment, an optical image is acquired by using an optical camera as the image acquisition apparatus 130. That is, the image acquisition apparatus 130 optically captures a subject, thereby acquiring an optical image. The configuration of the image acquisition apparatus 130 is not limited as long as the image acquisition apparatus 130 can acquire image information regarding a captured image. In the present exemplary embodiment, the image acquisition apparatus 130 is attached to the tubular lamp 122 and performs imaging in the radiation generation direction of the tubular lamp 122.

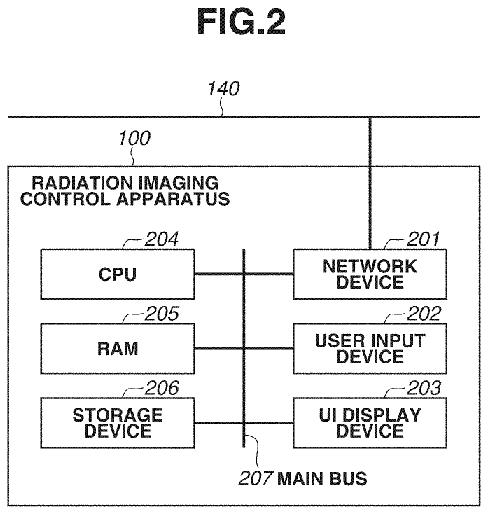

FIG. 2 is an example of the hardware configuration of the radiation imaging control apparatus 100 of the radiation imaging system according to the present exemplary embodiment.

The radiation imaging control apparatus 100 includes a network device 201 that connects to the network 140, a user input device 202 such as a keyboard that receives a user operation.

The radiation imaging control apparatus 100 also includes a user interface (UI) display device 203 such as a liquid crystal display that displays an operation screen and a radiation image, and a central processing unit (CPU) 204 that controls the entirety of the radiation imaging control apparatus 100.

The radiation imaging control apparatus 100 further includes a random-access memory (RAM) 205 that provides a work space for the CPU 204, and a storage device 206 that stores various control programs, a radiation image received from the radiation imaging apparatus 110, and image information received from the image acquisition apparatus 130.

The devices included in the radiation imaging control apparatus 100 are connected together by a main bus 207 and can transmit and receive data to and from each other.

Although the user input device 202 and the UI display device 203 are separate devices in the above description, an operation unit in which these devices are integrated together may be used.

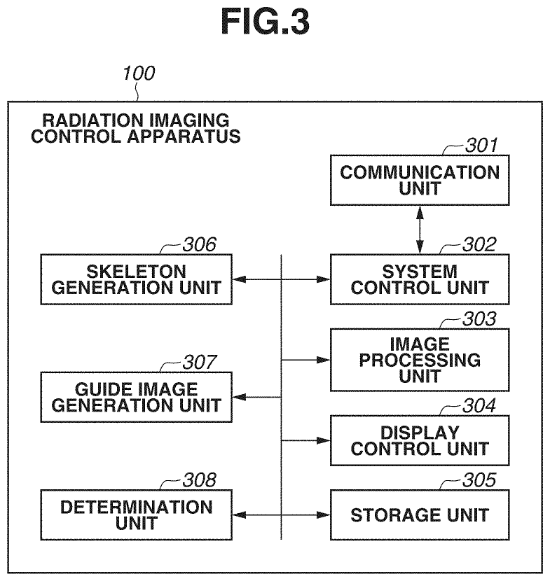

FIG. 3 is an example of the functional configuration of the radiation imaging control apparatus 100 of the radiation imaging system according to the present exemplary embodiment.

Function units illustrated in FIG. 3 are achieved by the CPU 204 of the radiation imaging control apparatus 100 loading a control program stored in the storage device 206 onto the RAM 205 and executing the control program.

The radiation imaging control apparatus 100 includes a communication unit 301, a system control unit 302, an image processing unit 303, a display control unit 304, a storage unit 305, a skeleton generation unit 306, a guide image generation unit 307, and a determination unit 308.

The communication unit 301 is software that controls the network device 201 to perform communication.

The system control unit 302 controls the image acquisition apparatus 130, acquires emission information regarding the radiation generating apparatus 120 and imaging information regarding the radiation imaging apparatus 110, and manages the states of these apparatuses via the communication unit 301. The system control unit 302 also acquires a radiation image from the radiation imaging apparatus 110 and an optical image from the image acquisition apparatus 130 via the communication unit 301.

The system control unit 302 is also a program that achieves the basic function of the radiation imaging control apparatus 100, and controls the operations of the units of the radiation imaging control apparatus 100.

The image processing unit 303 processes a radiation image acquired via the system control unit 302, thereby generating an image to be used by the radiation imaging control apparatus 100.

The display control unit 304 displays an image generated by the image processing unit 303 via the UI display device 203. The display control unit 304 also displays a guide image generated by the guide image generation unit 307 via the UI display device 203. The display control unit 304 also displays a past guide image generated from an image obtained by capturing a patient in the same posture in the past, via the UI display device 203. Further, the display control unit 304 reflects processing on an image indicated by the system control unit 302 and performs the process of switching screen display on the UI display device 203, based on an operation through the user input device 202.

The storage unit 305 stores a radiation image generated by the image processing unit 303, the imaging conditions (e.g., patient identification (ID), inspection ID, an inspection part, and an imaging direction) of the patient related to the radiation image, and emission information (e.g., a tube voltage and a tube current) regarding the radiation generating apparatus 120. The storage unit 305 also stores a guide image related to the radiation image and generated by the guide image generation unit 307, together with the radiation image, the imaging conditions, and the emission information.

The skeleton generation unit 306 estimates the skeleton and the joint angles of the patient in the optical image by using an optical image obtained from the image acquisition apparatus 130, and generates information indicating the skeleton and information indicating the joint angles. Specifically, the skeleton generation unit 306 estimates the skeleton and the joint angles of the patient in a current image by using a trained model obtained by performing machine learning using an optical image obtained by capturing a human body as input data and the skeleton and the joint angles of the captured human body as a label. The skeleton generation unit 306 then generates information indicating the skeleton and information indicating the joint angles based on the estimated skeleton and joint angles.

The “trained model” refers to a machine learning model according to a machine learning algorithm for, for example, deep learning using a support-vector machine or a neural network and refers to a machine learning model trained in advance using appropriate learning data. It is not that the trained model does not learn any further. The trained model can also perform additional learning. The learning data is composed of one or more groups of pairs of input data and output data (correct answer data). The trained model according to the present exemplary embodiment is constructed by performing supervised learning using a neural network and is trained on a set of input data (an optical image obtained by capturing a human body) and a label (the skeleton and the joint angles of the human body) as the learning data. That is, the skeleton generation unit 306 corresponds to an example of an estimation unit that estimates the skeleton and the joint angles of the subject from the first optical image using a trained model trained on a set of a plurality of optical images obtained by capturing a human body and information indicating a skeleton of the human body and information indicating a joint angle of the human body in the plurality of optical images. The machine learning algorithm and the learning data set used for learning are not limited to the above. Alternatively, for example, a model for estimating the skeleton and a model for estimating the joint angles may be different from each other. Although the skeleton generation unit 306 generates information indicating the skeleton and information indicating the joint angles in the present exemplary embodiment, the skeleton generation unit 306 only needs to be configured to generate at least one of information indicating the skeleton and information indicating the joint angles. Further, although the skeleton generation unit 306 generates information indicating the joint angles in the above description, the trained model may learn not only the joint angles but also the joint positions as a label. In such a case, the skeleton generation unit 306 can also similarly generate information indicating the joint positions.

The configuration of the skeleton generation unit 306 is not limited as long as the skeleton generation unit 306 can generate information indicating the skeleton of the patient and information indicating the joint angles of the patient in the optical image by using an optical image obtained from the image acquisition apparatus 130. For example, the skeleton generation unit 306 may acquire an optical image and position information including information regarding a distance from the image acquisition apparatus 130, acquire the coordinates of the joints of the patient, and apply the coordinates to human body data stored in advance, thereby generating information indicating the skeleton of the patient in the optical image and information indicating the joint angles of the patient.

The guide image generation unit 307 generates a guide image in which the information indicating the skeleton and the information indicating the joint angles are displayed in a superimposed manner on the optical image by using the information indicating the skeleton, the information indicating the joint angles generated by the skeleton generation unit 306, and an optical image obtained from the image acquisition apparatus 130. That is, the guide image generation unit 307 generates a first processed image including at least one of information indicating a skeleton of the subject and information indicating a joint angle of the subject related to the first optical image, based on a first optical image of the subject acquired at a first time. Thereafter, the guide image generation unit 307 instructs the display control unit 304 to display the guide image on a screen.

If the determination unit 308 determines that the patient has been captured in the same posture in the past, the guide image generation unit 307 acquires information regarding a guide image in the past regarding the patient that is stored in the storage unit 305, and generates a past guide image. That is, the guide image generation unit 307 generates a second processed image including at least one of information indicating the skeleton of the subject and information indicating the joint angle of the subject related to the second optical image, based on a second optical image of the subject acquired at a second time different from the first time. The information regarding the guide image in the past includes, for example, a past image, and information indicating the skeleton of the patient and information indicating the joint angles of the patient related to the past image. The past guide image refers to, for example, an image in which, on a past image, information indicating the skeleton and information indicating the joint angles related to the past image are superimposed. Thereafter, the guide image generation unit 307 instructs the display control unit 304 to display on the screen the guide image on which the past guide image is superimposed.

Based on the imaging conditions of the patient to be captured that are input in advance to the radiation imaging control apparatus 100 by the user, the determination unit 308 determines whether there is information matching any of the imaging conditions of the patient in the past in the storage unit 305. Although the determination unit 308 uses information regarding at least the patient ID, the inspection ID, the inspection part, and the imaging direction as the imaging conditions of the patient in the present exemplary embodiment, the configuration of the information is not limited as long as such information can be determined whether the information matches any of the imaging conditions of the patient in the past. In the present exemplary embodiment, if there is information matching a plurality of imaging conditions of the patient in the past, the determination unit 308 uses the latest information as the information matching any of the imaging conditions of the patient. The information to be used as the information matching any of the imaging conditions of the patient is not limited to the above, and may not be the latest information.

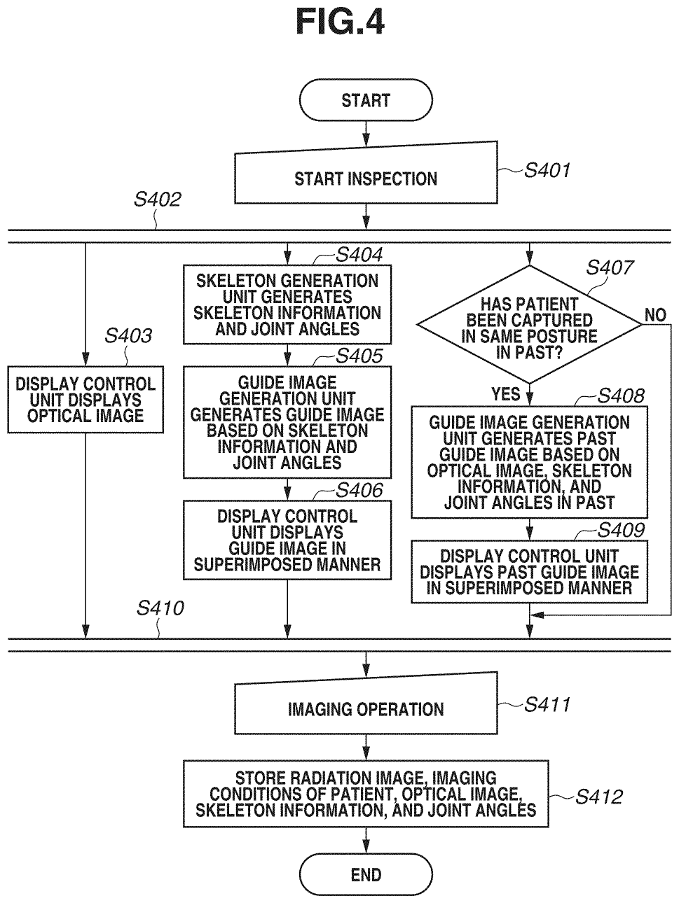

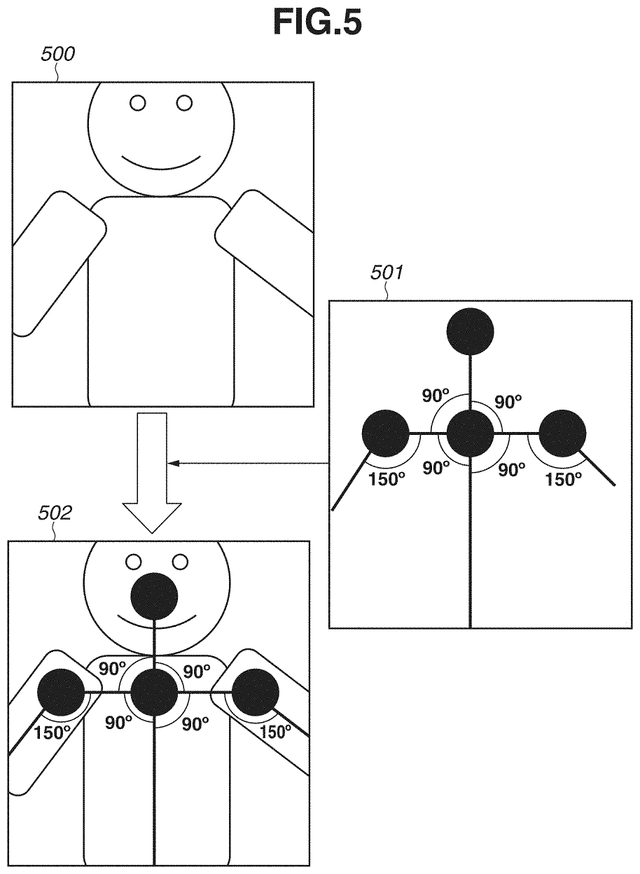

With reference to FIG. 4, a description is given of a display process method when the radiation imaging control apparatus 100 according to the present exemplary embodiment captures the patient. With reference to FIGS. 5 to 7, a description is given of examples of configurations regarding the display of an optical image, a guide image, and a past guide image.

FIG. 4 is a flowchart illustrating an example of a display processing process when the radiation imaging control apparatus 100 captures the patient.

In step S401, the system control unit 302 brings the radiation imaging control apparatus 100 into an inspection start state where the radiation imaging control apparatus 100 performs imaging control, based on a user operation. Specifically, the system control unit 302 transmits an instruction to prepare for imaging to the radiation imaging apparatus 110 via the communication unit 301, based on the imaging conditions of the patient for which an inspection instruction is given by a user operation. If the imaging preparation of the radiation imaging apparatus 110 is completed, the radiation imaging apparatus 110 transmits a preparation completion notification back to the radiation imaging control apparatus 100. After receiving the preparation completion notification, the system control unit 302 brings the radiation imaging control apparatus 100 into a state where the radiation imaging control apparatus 100 can perform imaging, so that the system control unit 302 will receive an imaging operation performed in step S411. The system control unit 302 transmits an instruction to start imaging to the image acquisition apparatus 130 via the communication unit 301. After receiving the imaging start instruction, the image acquisition apparatus 130 sequentially transmits an optical image acquired by the image acquisition apparatus 130 back to the radiation imaging control apparatus 100.

Between steps S402 and S410, the system control unit 302 executes serial parallel processing. That is, step S403, steps S404 to S406, steps S407 to S409, a control process other than these steps, and the reception of user control are performed. The processes between steps S402 and S410 are executed by the system control unit 302 until step S411 is executed, or the inspection is stopped by a user operation (not illustrated).

The processes between steps S402 and S410 do not necessarily need to be performed in parallel. The display device only needs to be able to display an optical image obtained by capturing the patient, a guide image generated from the optical image, and a past guide image generated from an optical image obtained by capturing the patient in the past.

For example, after the process of step S403 is executed, the processes of steps S404 to S406 may be executed, and then, the processes of steps S407 to S409 may be performed.

In step S403, the system control unit 302 displays the optical image acquired from the image acquisition apparatus 130 via the communication unit 301, on the UI display device 203 via the display control unit 304.

In step S404, the skeleton generation unit 306 generates information indicating the skeleton of the patient and information indicating the joint angles of the patient in the optical image, based on the optical image acquired via the system control unit 302.

In step S405, the guide image generation unit 307 generates a guide image, based on the information indicating the skeleton and the information indicating the joint angles generated by the skeleton generation unit 306.

In step S406, the display control unit 304 displays on the UI display device 203 a first superimposed image 502 in which the guide image as illustrated in FIG. 5 is superimposed on the optical image. That is, the display control unit 304 displays on a display unit the first processed image generated by superimposing on the first optical image at least one of the information indicating the skeleton of the subject and the information indicating the joint angle of the subject related to the first optical image obtained by optically capturing the subject.

A description is given of a configuration regarding the display of the optical image and the guide image displayed on the UI display device 203 in steps S403 to S406 with reference to FIG. 5.

Steps S403 to S406 are the process of displaying, on the UI display device 203, information indicating the skeleton and information indicating the joint angles in a superimposed manner on an optical image obtained by capturing in real time the patient that is being inspected.

An optical image 500 is an optical image acquired from the image acquisition apparatus 130 and displayed on the UI display device 203 in step S403. In the actual optical image, an object in the imaging range of the image acquisition apparatus 130 appears. For example, the radiation imaging apparatus 110 may be present behind the patient. However, for illustrative purposes, a diagram illustrating only body information regarding the patient is used as the optical image 500. Also as subsequent optical images and images related to the subsequent optical images, diagrams illustrating only body information regarding the patient are used, unless otherwise noted.

A guide image 501 is an image generated by the guide image generation unit 307 based on information indicating the skeleton of the patient and information indicating the joint angles of the patient generated by the skeleton generation unit 306. The guide image generation unit 307 instructs the display control unit 304 to display the guide image 501 in a superimposed manner on the optical image 500. Then, the display control unit 304 displays as a first superimposed image 502 the optical image 500 on which the guide image 501 is displayed in a superimposed manner. In the present exemplary embodiment, the joint angles of the patient generated by the skeleton generation unit 306 are displayed at the positions of the angles between the joint positions of a human body. The display method, however, is not limited as long as the method represents the joint angles that identify the locations of the joint positions of the human body. For example, character strings indicating position information regarding the joint angles may be displayed together with the joint angles.

The description returns to the flowchart in FIG. 4.

Steps S407 to S409 are the process of displaying, on the UI display device 203, information indicating the skeleton of the patient and information indicating the joint angles of the patient in a superimposed manner on an optical image obtained by capturing the patient under the same imaging conditions in the past.

In step S407, the determination unit 308 searches the storage unit 305, thereby determining whether there is an imaging condition in the past matching any of the imaging conditions of the patient for which the inspection instruction is given.

If it is determined in step S407 that the patient has been captured under the same imaging conditions in the past (Yes in step S407), the processing proceeds to step S407. In step S408, the guide image generation unit 307 acquires an optical image obtained based on the imaging conditions in the storage unit 305 and information indicating the skeleton and information indicating the joint angles related to the optical image and generates a second superimposed image 701.

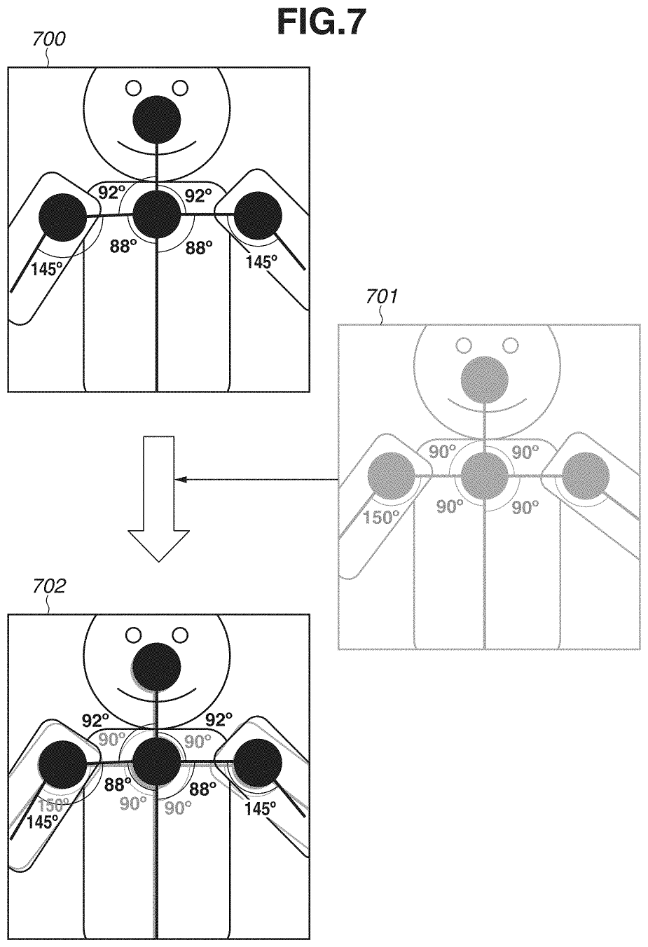

In step S409, the display control unit 304 displays on the UI display device 203, as illustrated in FIG. 7, the second superimposed image 701 as a past guide image in a superimposed manner on the optical image and the guide image. That is, the display control unit 304 displays on the display unit the second processed image generated by superimposing, on the second optical image, at least one of information indicating the skeleton of the subject and information indicating the joint angles of the subject that are stored in association with the second optical image captured in the past before the first optical image.

If it is not determined in step S407 that the patient has been captured under the same imaging condition in the past (No in step S407), the radiation imaging control apparatus 100 does not generate a past guide image, i.e., does not execute the processes of steps S408 and S409.

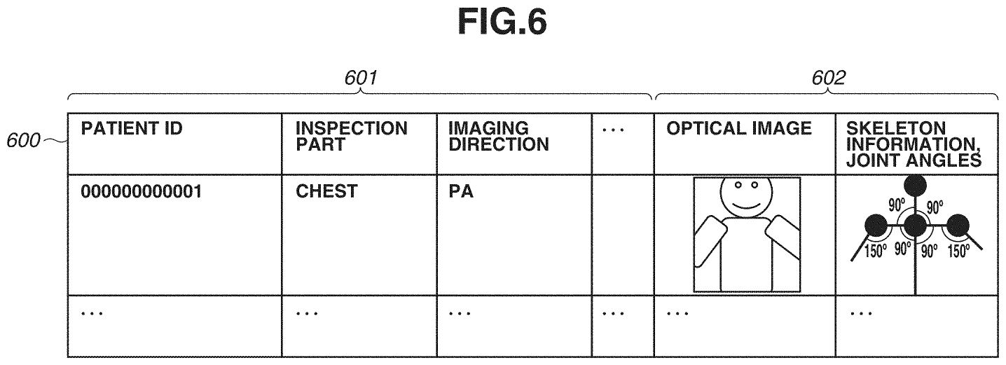

With reference to FIG. 6, a description is given of an example of the configuration of patient information stored in the storage unit 305 regarding the past guide image displayed on the UI display device 203 in steps S407 to S409. With reference to FIG. 7, an example of a configuration regarding the display of the past guide image is also described.

FIG. 6 is a diagram illustrating an example of the configuration of patient information stored in the storage unit 305. The storage unit 305 has a past patient imaging condition table 600. The past patient imaging condition table 600 includes an imaging condition portion 601 and a guide image information portion 602.

The imaging condition portion 601 stores, for example, a radiation image generated by the image processing unit 303, the imaging conditions of the patient related to the radiation image, and emission information regarding the radiation generating apparatus 120. Although the determination unit 308 uses information regarding at least, for example, the patient ID, the inspection ID, the inspection part, and the imaging direction as the imaging conditions of the patient in the present exemplary embodiment, the configuration of the information is not limited as long as the information can be used to determine whether the information matches any of the imaging conditions of the patient in the past. In the present exemplary embodiment, if there is information matching a plurality of imaging conditions of the patient in the past, the determination unit 308 uses the latest information as the information matching any of the imaging conditions of the patient. The information to be used as the information matching any of the imaging conditions of the patient is not limited to the above, and may not be the latest information.

The guide image information portion 602 stores an optical image acquired from the image acquisition apparatus 130 and information indicating the skeleton and information indicating the joint angles generated by the skeleton generation unit 306 when the radiation image in the imaging condition portion 601 is generated by the image processing unit 303. Although an optical image and information indicating the skeleton and information indicating the joint angles related to the optical image are stored in association with each other in the present exemplary embodiment, a configuration may be employed in which a guide image generated by the guide image generation unit 307 is also stored together with the optical image, the information indicating the skeleton, and the information indicating the joint angles, or only the guide image is stored. Although information indicating the skeleton and information indicating the joint angles are stored as image information in the present exemplary embodiment, a configuration may be employed in which only joint coordinate information in information indicating the skeleton is stored, and the guide image generation unit 307 generates information indicating the skeleton and information indicating the joint angles based on the joint coordinate information. That is, information indicating the skeleton and information indicating the joint angles do not necessarily need to be stored and displayed as image information, for example, as illustrated in FIG. 6, and may be stored and displayed simply as characters or numerical parameters.

Although the guide image generation unit 307 references the past patient imaging condition table 600 in the storage unit 305 in the present exemplary embodiment, the configuration of the past patient imaging condition table 600 is not limited as long as the determination in step S407 can be made. That is, the determination may be made using information on an information processing apparatus (not illustrated) having the past patient imaging condition table 600 via the network 140.

FIG. 7 is a diagram illustrating an example of a configuration regarding the display of the past guide image.

A first superimposed image 700 is an image generated similarly to the first superimposed image 502.

A second superimposed image 701 is an image generated as follows. In step S408, the guide image generation unit 307 acquires an optical image matching a predetermined imaging condition and information indicating the skeleton and information indicating the joint angles related to the optical image from the storage unit 305 and generates an image based on the optical image, the information indicating the skeleton, and the information indicating the joint angles. The guide image generation unit 307 instructs the display control unit 304 to display the second superimposed image 701 in a superimposed manner on the first superimposed image 700. Then, the display control unit 304 having received the display instruction displays as a third superimposed image 702 the first superimposed image 700 on which the second superimposed image 701 is displayed in a superimposed manner.

The description returns to the flowchart in FIG. 4 again.

In step S411, the user presses the exposure switch 121 of the radiation generating apparatus 120, thereby starting imaging. If the imaging is started, the radiation generating apparatus 120 causes the tubular lamp 122 to generate radiation. The radiation having passed through the patient is detected by the radiation imaging apparatus 110, and the radiation imaging apparatus 110 generates a radiation image. Thereafter, the radiation imaging apparatus 110 transmits the radiation image to the radiation imaging control apparatus 100. In parallel with such processing, the radiation generating apparatus 120 transmits emission information regarding the radiation imaging to the radiation imaging control apparatus 100.

In step S412, the system control unit 302 stores in the storage unit 305, for example, the radiation image, the imaging conditions of the patient related to the radiation image, and the emission information regarding the radiation generating apparatus 120. In parallel with the above processing, the system control unit 302 stores in the storage unit 305 an optical image when the imaging is performed and information indicating the skeleton and information indicating the joint angles related to the optical image.

As described above, the processing of the radiation imaging system according to the present exemplary embodiment is performed.

According to the above, the display control unit 304 displays an optical image acquired from the image acquisition apparatus 130 when an inspection is started, and a guide image generated based on information indicating the skeleton and information indicating the joint angles generated by the skeleton generation unit 306, in a superimposed manner on the UI display device 203. The display control unit 304 also determines a patient captured under the same imaging conditions in the past, displays an optical image obtained in the inspection in the past and a past guide image generated based on information indicating the skeleton and information indicating the joint angles related to the optical image in a superimposed manner on the UI display device 203.

Consequently, it is possible to display on a display device not only an optical image obtained by capturing a patient as an inspection target, but also a past image generated from an optical image captured in the past in the state where information indicating the skeleton and information indicating the joint angles are superimposed. Thus, an inspector can perform quantitative positioning based on information indicating the skeleton and information indicating the joint angles displayed in a superimposed manner on a past image and information indicating the skeleton and information indicating the joint angles displayed in a superimposed manner on a current image.

(First Variation) In the first exemplary embodiment, the display control unit 304 displays in a superimposed manner a current image on which information indicating the skeleton and information indicating the joint angles related to the current image are superimposed, and a past image on which information indicating the skeleton and information indicating the joint angles stored in association with the past image are superimposed, thereby improving positioning accuracy.

In contrast, in this variation, the display control unit 304 displays in a superimposed manner a current image obtained by changing pixel values based on information indicating the skeleton and information indicating the joint angles related to the current image, and a past image obtained by changing pixel values based on information indicating the skeleton and information indicating the angles of the joints stored in association with the past image.

More specifically, the skeleton generation unit 306 generates a guide image by changing the pixel values of pixels included in a current image so that information indicating the skeleton of the patient and information indicating the joint angles of the patient in the current image that are estimated similarly to the first exemplary embodiment can be visually confirmed. The skeleton generation unit 306 also generates a past guide image by changing the pixel values of pixels included in a past image so that information indicating the skeleton of the patient and information indicating the joint angles of the patient in the past image can be visually confirmed.

According to the above, similarly to the first exemplary embodiment, an inspector can perform quantitative positioning based on information indicating the skeleton and information indicating the joint angles displayed in a superimposed manner on a past image and information indicating the skeleton and information indicating the joint angles displayed in a superimposed manner on a current image.

(Second Variation)

In the first exemplary embodiment, the display control unit 304 displays in a superimposed manner a current image on which information indicating the skeleton and information indicating the joint angles related to the current image are superimposed, and a past image on which information indicating the skeleton and information indicating the joint angles stored in association with the past image are superimposed, thereby improving positioning accuracy.

In contrast, in this variation, the display control unit 304 displays in a superimposed manner only information indicating the skeleton and information indicating the joint angles related to a current image and information indicating the skeleton and information indicating the angles of the joints stored in association with a past image.

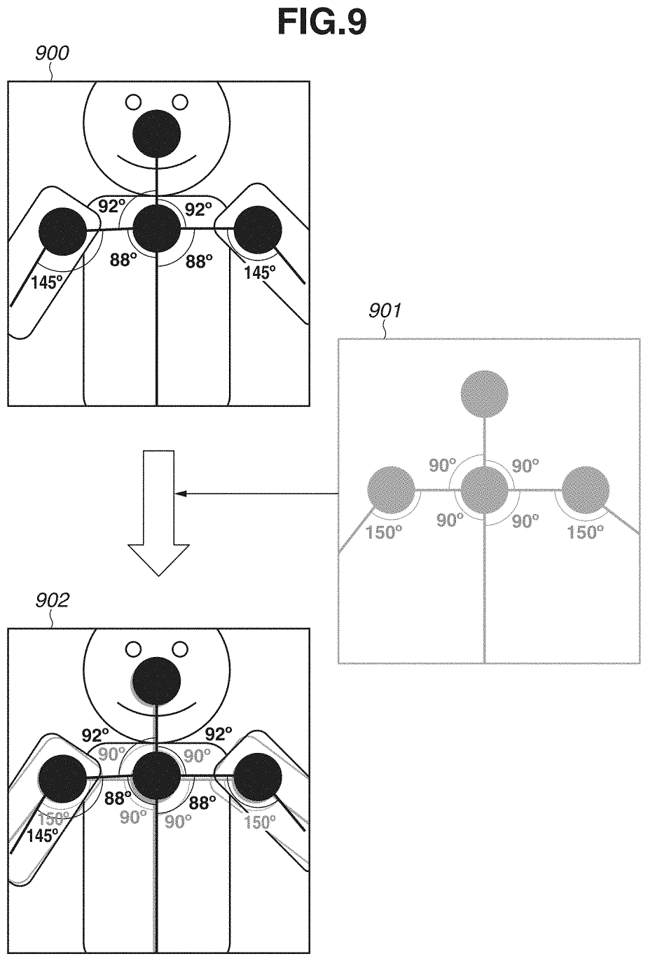

More specifically, the guide image 501 as illustrated in FIG. 5 and a past guide image 901 as illustrated in FIG. 9 are displayed in a superimposed manner.

According to the above, it is possible to perform positioning based on only information indicating the skeleton and information indicating the angles of the joints without displaying an optical image obtained by capturing a patient. Thus, it is possible to perform quantitative positioning in the interest of the privacy of the patient.

### Second Exemplary Embodiment

A radiation imaging system according to a second exemplary embodiment of the present invention will now be described.

In the configuration of the second exemplary embodiment, a process is added in which the radiation imaging control apparatus 100 makes changes based on the settings of the display contents of a guide image and a past guide image based on user settings.

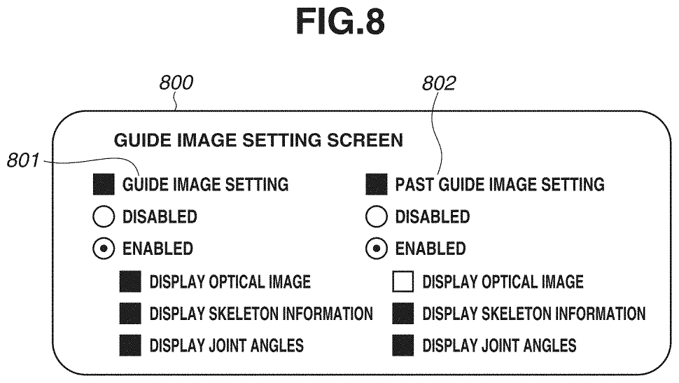

With reference to FIGS. 8 and 9, only the differences from the first exemplary embodiment are described below.

FIG. 8 is a diagram illustrating an example of the configuration of a guide image setting screen of the radiation imaging control apparatus 100 according to the present exemplary embodiment.

The guide image generation unit 307 additionally includes a guide image setting screen 800. The guide image setting screen 800 is displayed on the UI display device 203 according to a display instruction from the system control unit 302 (e.g., in a case where the user selects the guide image setting screen 800 through a system setting screen (not illustrated) by operating the radiation imaging control apparatus 100).

The guide image setting screen 800 includes a guide image setting portion 801 and a past guide image setting portion 802.

The guide image setting portion 801 includes a setting regarding a guide image generated based on an optical image and information indicating the skeleton and information indicating the joint angles related to the optical image. If the user sets the setting to disabled, the radiation imaging control apparatus 100 does not generate a guide image. Specifically, the radiation imaging control apparatus 100 does not execute the processes of steps S403 to S406 in FIG. 4. If the user sets the setting to enabled, the radiation imaging control apparatus 100 additionally sets the display of an optical image and information indicating the skeleton and information indicating the joint angles related to the optical image. Among items, only an item set to enabled by the user is displayed as an optical image and a guide image in the radiation imaging control apparatus 100. The description of the present exemplary embodiment continues on the assumption that the display of an optical image and information indicating the skeleton and information indicating the joint angles related to the optical image is enabled.

The past guide image setting portion 802 includes a setting regarding a past guide image generated based on an optical image matching a predetermined imaging condition among optical images captured in the past and information indicating the skeleton and information indicating the joint angles related to the optical image. If the user sets the setting to disabled, the radiation imaging control apparatus 100 does not generate a past guide image. Specifically, the radiation imaging control apparatus 100 does not execute the processes of steps S407 to S409 in FIG. 4. If the user sets this setting to enabled, the radiation imaging control apparatus 100 additionally sets the display of an optical image and information indicating the skeleton and information indicating the joint angles related to the optical image. Among items, only an item set to enabled by the user is displayed as a past guide image in the radiation imaging control apparatus 100. The description of the present exemplary embodiment continues on the assumption that the display of information indicating the skeleton and information indicating the joint angles is enabled.

If only the display of information indicating the joint angles is set to enabled, the locations and the positions of the joint angles may be additionally displayed. Specifically, for example, the joint angles and joint position information may be displayed together, or the locations of the parts of the joint angles may be highlighted by surrounding the locations with figures.

FIG. 9 is a diagram illustrating an example of a configuration regarding the display of the past guide image. A configuration regarding the display of the optical image and the guide image is similar to that in the first exemplary embodiment, and therefore is not described. The setting of the guide image in the radiation imaging control apparatus 100 in this case is based on the setting on the guide image setting screen 800 illustrated in FIG. 8.

A superimposed image 900 is an image generated by the guide image generation unit 307 based on the setting on the guide image setting screen 800 in steps S403 to S406 in the first exemplary embodiment.

A past guide image 901 is an image generated as follows. In step S408 in the first exemplary embodiment, the guide image generation unit 307 acquires information indicating the skeleton and information indicating the joint angles from the storage unit 305 based on the setting on the guide image setting screen 800 and generates an image. The guide image generation unit 307 instructs the display control unit 304 to display the past guide image 901 in a superimposed manner on the superimposed image 900. The display control unit 304 then displays as a past guide superimposed image 902 the superimposed image 900 on which the past guide image 901 is displayed in a superimposed manner.

Based on the above, in the second exemplary embodiment, the radiation imaging control apparatus 100 controls the display of a guide image and a past guide image based on the settings of the display contents of a guide image and a past guide image based on user settings. Consequently, the user can selectively display information appropriate for reproducing positioning.

### Third Exemplary Embodiment

A radiation imaging system according to a third exemplary embodiment of the present invention will now be described.

In the configuration of the third exemplary embodiment, a process in which the radiation imaging control apparatus 100 generates a guide image based on three-dimensional skeleton information and joint angle information regarding the patient is added.

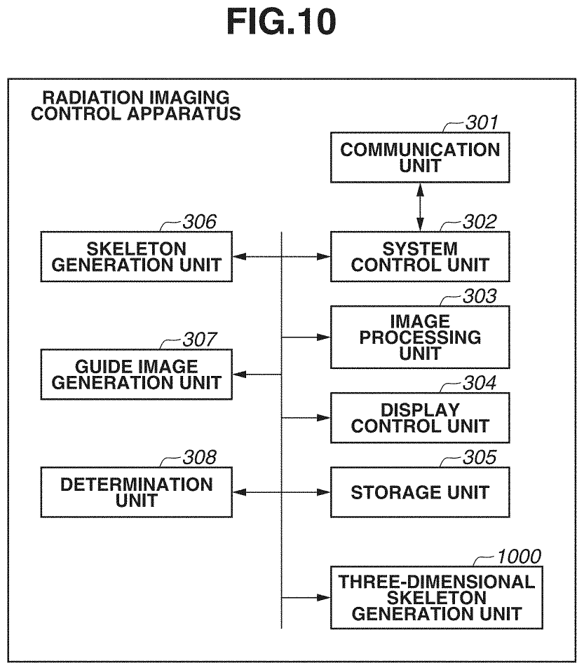

With reference to FIGS. 10 to 12, only the differences from the first exemplary embodiment are described below.

FIG. 10 is an example of the configuration of the radiation imaging control apparatus 100 of the radiation imaging system according to the present exemplary embodiment. The radiation imaging control apparatus 100 additionally includes a three-dimensional skeleton generation unit 1000.

The three-dimensional skeleton generation unit 1000 generates information indicating the three-dimensional skeleton and information indicating the three-dimensional joint angles. As the generation method, a technique similar to that in the first exemplary embodiment can be used.

For example, the three-dimensional skeleton generation unit 1000 estimates the skeleton and the joint positions of a human body in a current image by using a trained model obtained by performing machine learning using an optical image obtained by capturing a human body as input data and the three-dimensional skeleton and the three-dimensional joint angles of the captured human body as a label.

In the present exemplary embodiment, a three-dimensional image used as the input data may be generated by, for example, combining two-dimensional images captured from different angles by a plurality of optical cameras. Alternatively, the three-dimensional image used as the input data may be generated from two-dimensional images captured while changing the angle of a single optical camera by driving the single optical camera. That is, in the present exemplary embodiment, the method for acquiring a three-dimensional image is not limited as long as the method can acquire a three-dimensional image obtained by capturing a subject.

The three-dimensional skeleton generation unit 1000 then generates information indicating the three-dimensional skeleton and information indicating the three-dimensional joint angles based on the estimated skeleton and joint positions. Information generated in the present exemplary embodiment may be generated based on a two-dimensional optical image, or may be generated based on a three-dimensional optical image.

The configuration of the three-dimensional skeleton generation unit 1000 is not limited as long as the three-dimensional skeleton generation unit 1000 can generate information indicating the three-dimensional skeleton and information indicating the three-dimensional joint angles. For example, the three-dimensional skeleton generation unit 1000 may acquire an optical image and position information including information regarding a distance from the image acquisition apparatus 130, acquire the coordinates of the joints of the patient, and apply the coordinates to human body data saved in advance, thereby generating information indicating the three-dimensional skeleton of the patient and information indicating the three-dimensional joint angles of the patient in the optical image.

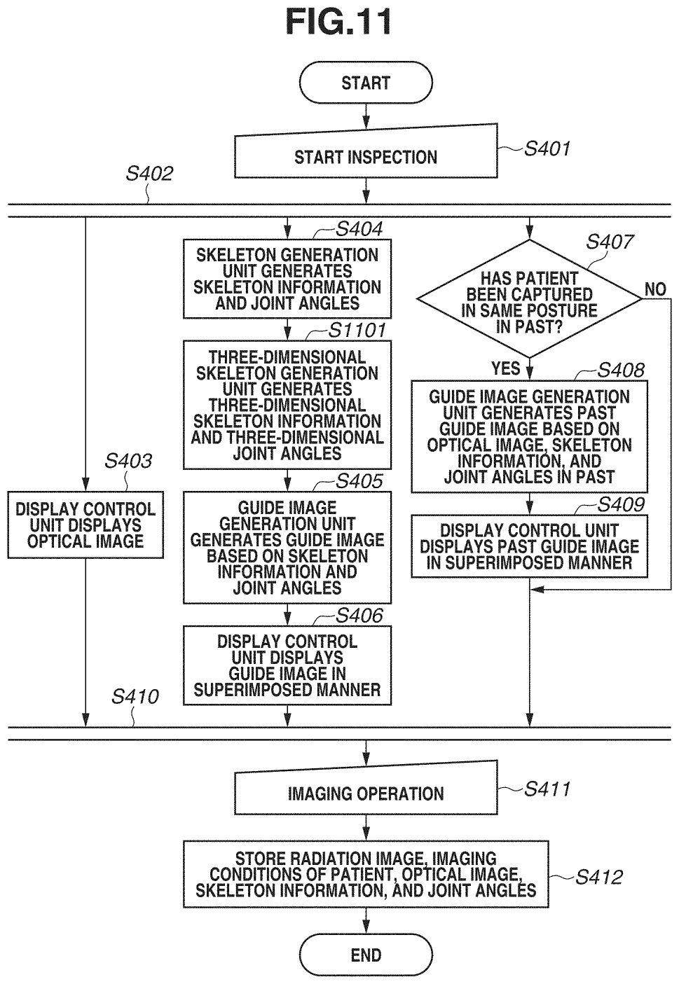

FIG. 11 is a flowchart illustrating an example of a display processing process when the radiation imaging control apparatus 100 captures the patient according to the present exemplary embodiment.

In step S1101, the three-dimensional skeleton generation unit 1000 generates information indicating the three-dimensional skeleton and information indicating the three-dimensional joint angles by using the information indicating the skeleton and the information indicating the joint angles generated by the skeleton generation unit 306. In the subsequent step S405, the guide image generation unit 307 then generates a guide image based on the information indicating the three-dimensional skeleton and the information indicating the three-dimensional joint angles generated by the three-dimensional skeleton generation unit 1000. Then, in the subsequent step S406, the guide image is displayed in a superimposed manner on the optical image on the UI display device 203 via the display control unit 304.

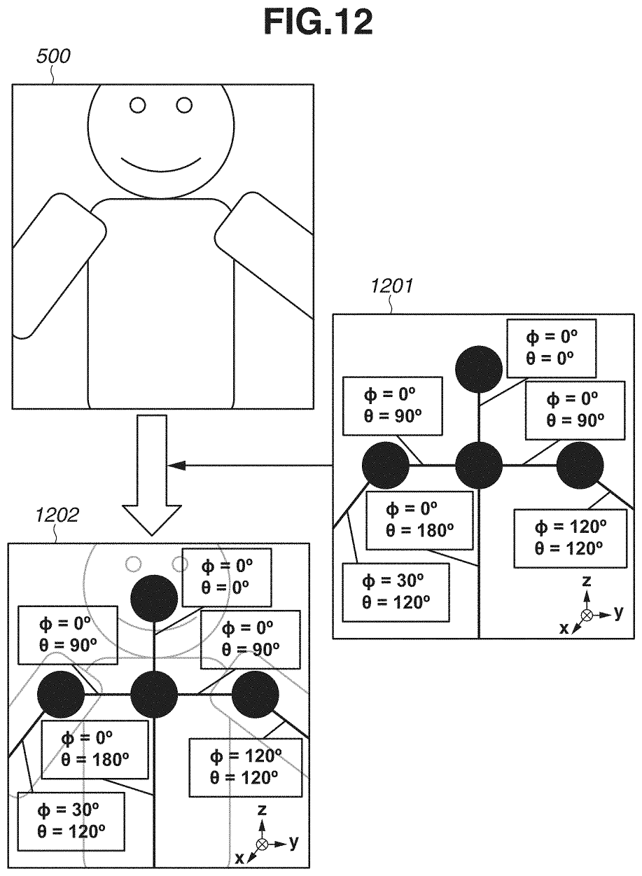

A description is given of a configuration regarding the display of the optical image and the guide image displayed on the UI display device 203 in steps S403 to S406 and step S1101 with reference to FIG. 12. A configuration regarding the display of the past guide image is similar to that in the first exemplary embodiment, and therefore is not described. That is, in step S408, information indicating the three-dimensional skeleton and information indicating the three-dimensional joint angles generated by the three-dimensional skeleton generation unit 1000 and stored in association with a past image are used to generate a past guide image.

A three-dimensional guide image 1201 is an image generated by the guide image generation unit 307 based on information indicating the three-dimensional skeleton of the patient and information indicating the three-dimensional joint angles of the patient generated by the three-dimensional skeleton generation unit 1000 in step S1101. The guide image generation unit 307 instructs the display control unit 304 to display the three-dimensional guide image 1201 in a superimposed manner on the optical image 500. The display control unit 304 then displays as a guide image superimposed display image 1202 the optical image 500 on which the three-dimensional guide image 1201 is displayed in a superimposed manner.

In the present exemplary embodiment, information indicating the three-dimensional joint angles of the patient generated by the three-dimensional skeleton generation unit 1000 is represented by displaying character strings in a three-dimensional polar coordinate format. That is, an angle θ between a z-axis and information indicating the skeleton in a direction from inside to outside at the joint position of a human body, and an angle tri between an x-axis and information indicating the skeleton arranged on an xy-plane are displayed. The method, however, is not limited to the above as long as the method represents the three-dimensional joint angles.

Based on the above, in the third exemplary embodiment, the radiation imaging control apparatus 100 generates a guide image, based on information indicating the three-dimensional skeleton and information indicating the three-dimensional joint angles. The user can thereby reference information indicating the three-dimensional skeleton and information indicating the three-dimensional joint angles. Thus, it is possible to further improve the reproducibility of the positioning of the posture of the patient by the user.

### Fourth Exemplary Embodiment

A radiation imaging system according to a fourth exemplary embodiment of the present invention will now be described.

In the fourth exemplary embodiment, the radiation imaging control apparatus 100 is configured to show the patient an image displayed on the UI display device 203, whereby the patient themselves can improve the reproducibility of their posture.

Specifically, the display control unit 304 additionally has a control function for displaying an optical image, a superimposed display image, or a past superimposed display image on the UI display device 203 in a horizontally flipped manner (not illustrated). In the processes performed in steps S402 to S410 illustrated in FIG. 4, the user sets the control function to enabled and presents the UI display device 203 to the patient.

Based on the above, the patient themselves can confirm the superimposed display of a current image, a guide image generated based on information indicating the skeleton of the patient and information indicating the joint angles of the patient related to the current image, a past image, and a past guide image generated based on information indicating the skeleton and information indicating the joint angles related to the past image. Consequently, the user can improve the reproducibility of the positioning of the posture of the patient, and the patient themselves can also improve the reproducibility of their posture.

The present invention is not limited to the above exemplary embodiments, and can be changed and modified in various ways without departing from the spirit and the scope of the present invention. Thus, the following claims are appended to publicize the scope of the present invention.

According to the present invention, in imaging using a radiation imaging system, it is possible to improve the reproducibility of the positioning of the posture of a patient.

### OTHER EMBODIMENTS

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. A radiation imaging system for imaging a subject using radiation comprising:
a first generation unit configured to generate, based on a first optical image of the subject acquired at a first time, a first processed image including at least one of information indicating a skeleton of the subject and information indicating a joint angle of the subject related to the first optical image;
a second generation unit configured to generate, based on a second optical image of the subject acquired at a second time different from the first time, a second processed image including at least one of information indicating the skeleton of the subject and information indicating the joint angle of the subject related to the second optical image; and
a display control unit configured to display the first processed image and the second processed image in a superimposed manner on a display unit.

2. The radiation imaging system according to claim 1,
wherein the first generation unit generates the first processed image by superimposing at least one of the information indicating the skeleton and the information indicating the joint angle on the first optical image, and
wherein the second generation unit generates the second processed image by superimposing at least one of the information indicating the skeleton and the information indicating the joint angle on the second optical image.

3. The radiation imaging system according to claim 1,
wherein the first generation unit generates the first processed image by changing a pixel value of a pixel included in the first optical image so that at least one of information indicating a position of the skeleton of the subject and information indicating the joint angle of the subject can be visually confirmed in the first optical image, and
wherein the second generation unit generates the second processed image by changing a pixel value of a pixel included in the second optical image so that at least one of information indicating the position of the skeleton of the subject and information indicating the joint angle of the subject can be visually confirmed in the second optical image.

4. The radiation imaging system according to claim 1, further comprising a storage unit configured to store in a storage portion at least one of the first optical image, the information indicating the skeleton and the information indicating the joint angle related to the first optical image, and the first processed image.

5. The radiation imaging system according to claim 4, wherein the storage unit stores in the storage portion the first optical image, the information indicating the skeleton and the information indicating the joint angle related to the first optical image, and the first processed image.

6. The radiation imaging system according to claim 1, further comprising an estimation unit configured to estimate the skeleton and the joint angle of the subject from the first optical image,
wherein the first generation unit generates the first processed image based on at least one of information indicating the skeleton of the subject generated based on the skeleton and the joint angle estimated by the estimation unit and information indicating the joint angle of the subject.

7. The radiation imaging system according to claim 6,
wherein the skeleton and the joint angle of the subject estimated by the estimation unit are three-dimensional information, and
wherein the first generation unit generates the first processed image based on at least one of information indicating a three-dimensional skeleton of the subject and information indicating a three-dimensional joint angle of the subject that are generated based on the three-dimensional skeleton and the three-dimensional joint angle estimated by the estimation unit.

8. The radiation imaging system according to claim 6, wherein the estimation unit estimates the skeleton and the joint angle of the subject from the first optical image using a learning model trained on a set of a plurality of optical images obtained by capturing a human body and information indicating a skeleton of the human body and information indicating a joint angle of the human body in the plurality of optical images.

9. The radiation imaging system according to claim 1, further comprising a determination unit configured to determine whether there is an optical image obtained based on a predetermined imaging condition among optical images captured in the past before the first optical image,
wherein the second generation unit generates the second processed image by superimposing at least one of information indicating the skeleton of the subject and information indicating the joint angle of the subject that are stored in association with the second optical image obtained based on the predetermined imaging condition among the optical images captured in the past before the first optical image, on the second optical image.

10. The radiation imaging system according to claim 9, wherein the predetermined imaging condition includes information regarding patient identification (ID), inspection ID, an inspection part, or an imaging direction.

11. The radiation imaging system according to claim 1, wherein in a case where the first generation unit generates the first processed image based on the information indicating the skeleton, the second generation unit generates the second processed image based on information indicating the skeleton stored in association with the second optical image, and in a case where the first generation unit generates the first processed image based on the information indicating the joint angle, the second generation unit generates the second processed image based on information indicating the joint angle stored in association with the second optical image.

12. The radiation imaging system according to claim 1,
wherein the first generation unit generates the first processed image based on the information indicating the skeleton of the subject and the information indicating the joint angle of the subject, and
wherein the second generation unit generates the second processed image based on information indicating the skeleton of the subject and information indicating the joint angle of the subject that are stored in association with the second optical image.

13. The radiation imaging system according to claim 1, further comprising a reception unit configured to receive selection of a setting regarding generation of the first processed image and the second processed image from a user,
wherein the display control unit displays information based on the setting selected by the reception unit on the display unit.

14. The radiation imaging system according to claim 13,
wherein the first generation unit generates the first processed image based on the setting received by the reception unit, and
wherein the second generation unit generates the second processed image based on the setting received by the reception unit.

15. The radiation imaging system according to claim 13, wherein the reception unit receives a setting of whether to display at least one of the first optical image, the information indicating the skeleton, and the information indicating the joint angle on the display unit.

16. The radiation imaging system according to claim 1, wherein the display control unit displays the first optical image and the first processed image and the second processed image on the display unit in a horizontally flipped manner.

17. A radiation imaging system for imaging a subject using radiation comprising:
a display control unit configured to display a first guide image generated based on at least one of information indicating a skeleton of the subject and information indicating a joint angle of the subject related to a first optical image of the subject acquired at a first time, and a second guide image generated based on at least one of information indicating the skeleton of the subject and information indicating the joint angle of the subject related to a second optical image acquired at a second time different from the first time, in a superimposed manner on a display unit.

18. An image processing apparatus comprising:
a first generation unit configured to generate, based on a first optical image of a subject acquired at a first time, a first processed image including at least one of information indicating a skeleton of the subject and information indicating a joint angle of the subject in the first optical image;
a second generation unit configured to generate a second optical image of the subject acquired at a second time different from the first time, and a second processed image including at least one of information indicating the skeleton of the subject and information indicating the joint angle of the subject in the second optical image; and
a display control unit configured to display the first processed image and the second processed image in a superimposed manner on a display unit.

19. A radiation imaging method for imaging a subject using radiation comprising:
based on a first optical image of the subject acquired at a first time, generating a first processed image including at least one of information indicating a skeleton of the subject and information indicating a joint angle of the subject related to the first optical image;
based on a second optical image of the subject acquired at a second time different from the first time, generating a second processed image including at least one of information indicating the skeleton of the subject and information indicating the joint angle of the subject related to the second optical image; and
displaying the first processed image and the second processed image in a superimposed manner on a display unit.

20. A non-transitory computer readable storage medium storing a program for causing a computer to execute the radiation imaging method according to claim 19.
